(19) United States
(12) Patent Application Publication
(10) Pub. No.: US 2010/0312090 A1
(43) Pub. Date: Dec. 9, 2010

(54) ATHEROSCLEROSIS RISK ASSESSMENT BY PROJECTED VOLUMES AND AREAS OF PLAQUE COMPONENTS

(75) Inventors: William S. Kerwin, Seattle, WA (US); Dongxiang Xu, Seattle, WA (US); Chun Yuan, Bellevue, WA (US)

Correspondence Address: Law Offices of Ronald M. Anderson, 600 108th Ave NE, Suite 507, Bellevue, WA, 98004, US

(73) Assignee: University of Washington Center for Commercialization, Seattle, WA (US)

(21) Appl. No.: 12/783,327

(22) Filed: May 19, 2010

Related U.S. Application Data
(60) Provisional application No. 61/184,700, filed on Jun. 5, 2009.

(51) Int. Cl. A61B 5/055; A61B 8/00; A61B 5/00; G06K 9/00

(52) U.S. Cl. 600/410; 600/437; 600/300; 382/128

(57) ABSTRACT

A novel technique directed toward risk assessment of a patient's plaque vulnerability, wherein clinical events may be caused by internal plaque components affecting a lumen within an artery. A surface area projection or shadow of one or more plaque components onto a lumen can be measured and assessed. Optionally, a total volume projection onto the lumen can also be measured and assessed to refine the determination of risk to a patient and to monitor the progression of atherosclerosis over time.

### RELATED APPLICATIONS

This application is based on a prior copending provisional application Ser. No. 61/184,700, filed on Jun. 5, 2009, the benefit of the filing date of which is hereby claimed under 35 U.S.C. §119(e).

### GOVERNMENT RIGHTS

This invention was made with U.S. government support under RO1HL56874 awarded by the National Institutes of Health. The U.S. government has certain rights in the invention.

### BACKGROUND

Atherosclerosis is the disease responsible for most heart attacks and strokes, and thus, it causes more deaths and disabilities than any other disease. Atherosclerosis is characterized by the buildup of plaque within the interior lining of arteries. At present, the risk posed by a given atherosclerotic plaque is evaluated as a function of the amount of blockage it causes within a vessel lumen (i.e., the interior channel through which blood flows). This measurement is called stenosis.

Unfortunately, although stenosis is the clinical standard for making this type of evaluation, it is a poor predictor of risk. Accumulating evidence increasingly points to characteristics of the plaque forming within vessels as being more important as predictors of the likely development of clinical complications of atherosclerosis than stenosis. What is lacking, however, is a clinically viable approach for characterizing risk due to these plaque characteristics.

Therefore, it would be desirable to develop a methodology that utilizes non-invasive medical imaging to identify plaque substructures, and subsequently compile that information into a single quantity that is better correlated with the risk of developing clinical complications, than is stenosis.

### SUMMARY

As discussed below, an exemplary method for assessing a risk of atherosclerosis in an artery, based on a plurality of images of the artery, includes the step of selecting a plaque component in the plurality of images. A dimension of the plaque component is then projected onto a circumference of a lumen within the artery. Also, a surface area of the plaque component is projected along a longitudinal axis of the lumen. Finally, the risk of atherosclerosis in the artery is assessed as a function of the dimension of the plaque component projected onto the circumference of the lumen, and the surface area of the plaque component projected along the longitudinal axis of the lumen.

The method can further include the step of determining a region of the plaque component. The region of the plaque component is then projected onto a surface of the lumen to define a corresponding volume. If this option is included, the risk of atherosclerosis can also be based on the corresponding volume of the region of the plaque component projected onto the surface of the lumen.

The step of projecting the dimension of the plaque component onto the circumference of the lumen can include the step of identifying boundaries of the artery, and lines of thickness between an internal boundary of a wall of the artery and an outer boundary of the wall of the artery. Specific lines of thickness that intersect the plaque component are determined, and points where each specific line of thickness intersects the circumference of the lumen are marked. A length along the circumference resulting from the step of projecting the dimension of the plaque component is determined, to encompass the points marked on the circumference of the lumen.

The step of projecting the surface area of the plaque component along a longitudinal axis of the lumen includes the step of determining a thickness of each cross-sectional image in which the lumen is intersected by the lines of thickness. A projected area of the plaque component for each cross-sectional image intersected by the lines of thickness is next determined. For each cross-sectional image intersected by the lines of thickness, a projected length of the plaque component is multiplied by the thickness of the cross-sectional image to determine a projected area for each such cross-sectional image. Further, a sum of all projected areas for the cross-sectional images is determined.

The step of projecting the region of the plaque component onto the surface of the lumen to define the corresponding volume can include the step of determining a maximum line of thickness for each cross-sectional image in which the plaque component is disposed. Next, an average maximum line of thickness is determined based on the maximum line of thickness that was determined; the average maximum line of thickness is set equal to a maximum thickness. A projected volume of the region of the plaque component is then determined by multiplying the surface area projected along the longitudinal axis of the lumen by the maximum thickness.

The method can further include the step of producing the images by using at least one of several different imaging approaches, including imaging the artery to form successive cross-sectional images along a longitudinal extent of the artery using magnetic resonance (MR) imaging, or computed tomography imaging, or ultrasound imaging. If MR imaging is used, different MR acquisition parameters can be employed for creating a plurality of sets of MR images of the artery, to achieve different contrast weightings for each set of MR images, for use in assessing the risk of atherosclerosis in the artery.

The step of selecting the plaque component can include the step of selecting either a lipid-rich necrotic core, a calcification, or a hemorrhage in a wall of the artery.

Another aspect of the present approach is directed to a non-transitory medium storing machine readable instructions that are executable by a computing device to facilitate assessing a risk of atherosclerosis in an artery, based on a plurality of images of the artery. When thus executed, the machine readable instructions are operable to carry out a plurality of functions that are generally consistent with the steps of the method discussed above. Similarly, yet another aspect is directed to a system for use in automatically assessing a risk of atherosclerosis in an artery, based on a plurality of images of the artery. The system includes a memory in which are stored machine instructions, a user input device, a display on which text and graphics are displayed, and a hardware processor that is coupled to the memory, the user input device, and the display. The processor executes the machine instructions stored in memory to carry out a plurality of functions that are generally consistent with the steps of the method discussed above.

This application specifically incorporates herein by reference, the disclosure and drawings of the patent application identified above as a related application.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1A is an exemplary cross-sectional image of a diseased, atherosclerotic artery;

FIG. 1B illustrates an axes convention superimposed on a three-dimensional view of a segment of an artery;

FIG. 2 is an overall flowchart of the steps carried out in an exemplary embodiment of the method;

FIG. 3 is a flowchart of exemplary steps carried out in determining a projected length of a selected plaque component on a vessel lumen;

FIG. 4 is a flowchart of exemplary steps carried out in determining a projected surface area of a selected plaque component onto a vessel lumen;

FIG. 5 is a flowchart of exemplary steps carried out in determining a projected volume of a selected plaque component relative to a vessel lumen;

FIG. 6A is an example of an MR image taken with a T1 contrast weighting;

FIG. 6B is an example of an MR image taken with a T2 contrast weighting;

FIG. 6C is an example of an MR image taken with a proton density contrast weighting;

FIG. 6D is an example of an MR image taken with a time-of-flight contrast weighting;

FIG. 7A illustrates how “lines of thickness” between inner and outer boundaries of a vessel wall are determined;

FIG. 7B illustrates how “lines of thickness” between inner and outer boundaries of a vessel wall that intersect a plaque component of interest are determined;

FIG. 8 illustrates the projection of the area of a plaque component onto the surface of a vessel lumen;

FIGS. 9A-9C illustrate three different plaque components that yield a different surface area once projected onto a vessel lumen, yet are the same size; and

FIG. 10 is a functional block diagram of an exemplary embodiment of a system used to produce projected surface area and projected volume measurements, in accord with the novel approach disclosed herein.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein. Further, it should be understood that any feature of one embodiment disclosed herein can be combined with one or more features of any other embodiment that is disclosed, unless otherwise indicated.

### Utility

The following discussion is directed to a novel approach that is useful to assess the risks associated with plaque vulnerability. Based on an analysis of the plaque components in a patient's atherosclerotically diseased arteries and vessel morphology, values of surface area and volume as a function of a selected plaque component's projection onto and into a vessel lumen, can respectively be determined. The value of surface area is also referred to herein as “plaque coverage” (PC). This measurement reflects the comprehensive impact to a vessel lumen due to changes in either plaque size or its proximity during plaque progression. The value of the projected volume for a plaque component is also of interest because of the effect of the total amount of material subject to a force from the lumen. These values correlate with a higher risk of incidents of negative health consequences, such as strokes, and transient ischemic attacks that lead to disabilities and fatalities, if the plaque comes into direct contact with a vessel lumen. In other words, as described below, an exemplary risk assessment tool and method of its use produce indices that can indicate the likelihood that a fibrous matrix will fissure, causing adverse effects on a patient's health.

In general, the risk of fissuring is associated with the size of the surface area of fibrous material that separates internal plaque components from the vessel lumen. An analogy can be made to determining how likely it is that a bridge will collapse, based on the length of its span. The risk of fissuring is also associated with the thickness of the plaque materials behind a fibrous area. In the bridge analogy, this thickness determination is analogous to determining how high a material is stacked on top a bridge, since an excessive height of stacked material can collapse the bridge.

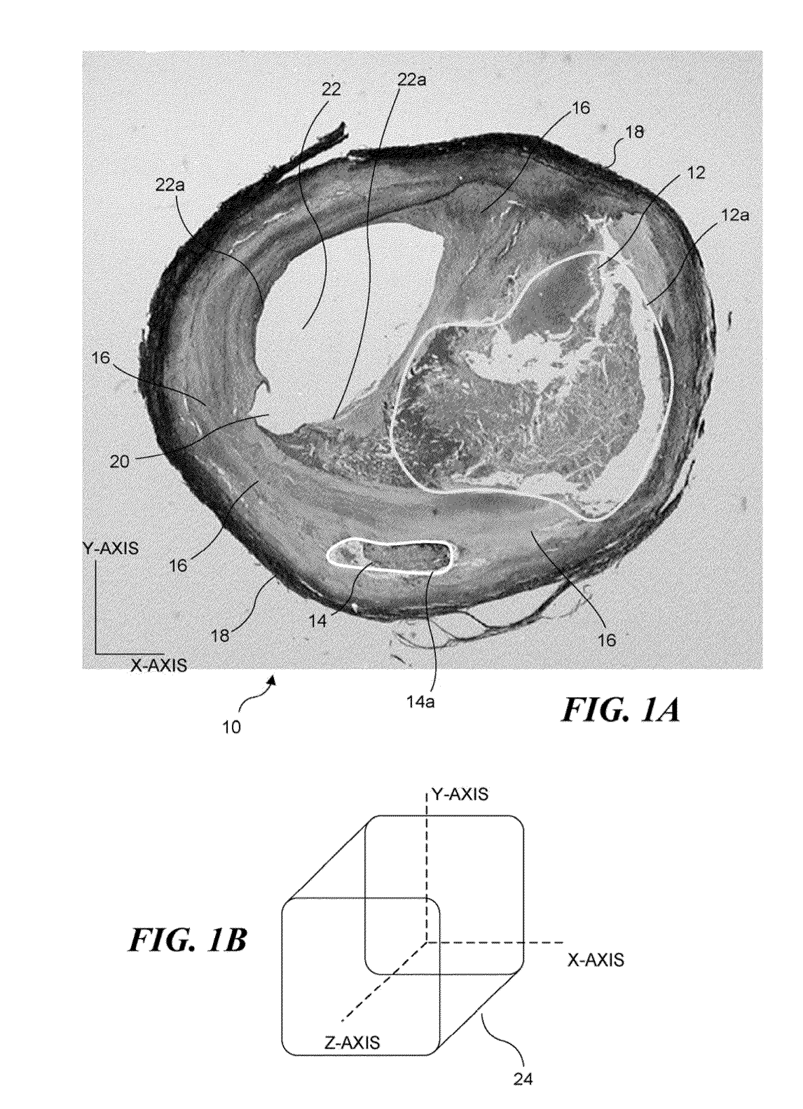

The usefulness of such a tool and its method of use is also evident from an inspection of FIG. 1A. In discussing FIG. 1A, it should be understood that FIG. 1B illustrates an axes convention used herein, where a segment of an artery 24 is illustrated, having a longitudinal axis extending generally along the Z axis, and orthogonal X and Y axes extending generally transversely relative to the artery. FIG. 1A is an exemplary cross-section of an artery 10 illustrating atherosclerotic plaque components within a fibrous matrix. Applying the axes convention of FIG. 1B, it will be understood that FIG. 1A is a slice or cross-sectional view in the XY plane of a segment of atherosclerotic artery 10. In other words, this cross-sectional image is acquired perpendicular to the Z axis or longitudinal axis of the artery.

Returning to FIG. 1A, atherosclerotic artery 10 is characterized by a buildup of internal plaque components, including a lipid-rich, necrotic core (LRNC) 12, a calcification 14, and a hemorrhage (not shown) embedded within a fibrous matrix 16. Fibrous matrix 16 comprises a matrix of fibrous tissue and smooth muscle cells. The internal plaque components occupy distinct regions or volumes bounded by a LRNC boundary 12a and a calcification boundary 14a, all within an artery outer wall boundary 18. The internal plaque components are generally separated from a vessel lumen 22 by the fibrous matrix. Internal plaque components that are exposed directly to the interior vessel lumen, for example, through fissuring of the fibrous matrix, account for the overwhelming majority of clinical events. In the example shown in FIG. 1A, a fissure 20 has formed in a boundary 22a of vessel lumen 22, indicating that the vessel lumen is now vulnerable to direct contact with any plaque components comprising the fibrous matrix. It must be emphasized that successive cross-sectional slices of the artery will be produced in a similar manner along the length of the artery that is being evaluated for risk. An analysis of this patient's atherosclerotic artery with respect to its composition and vessel morphology using the present novel approach can help to inform the patient of the risk and the preventative measures that might be taken to potentially reduce an undesired clinical event, such as stroke and transient ischemic attacks from occurring.

The exemplary embodiments described herein are directed toward a vessel lumen of an atherosclerotic carotid artery. However, those skilled in the art will recognize that the present novel approach is not intended to be limited to the described embodiments and not limited in application to a carotid artery, but can also be applied to other vessels, or to the aorta and coronary arteries.

### Exemplary Method Steps

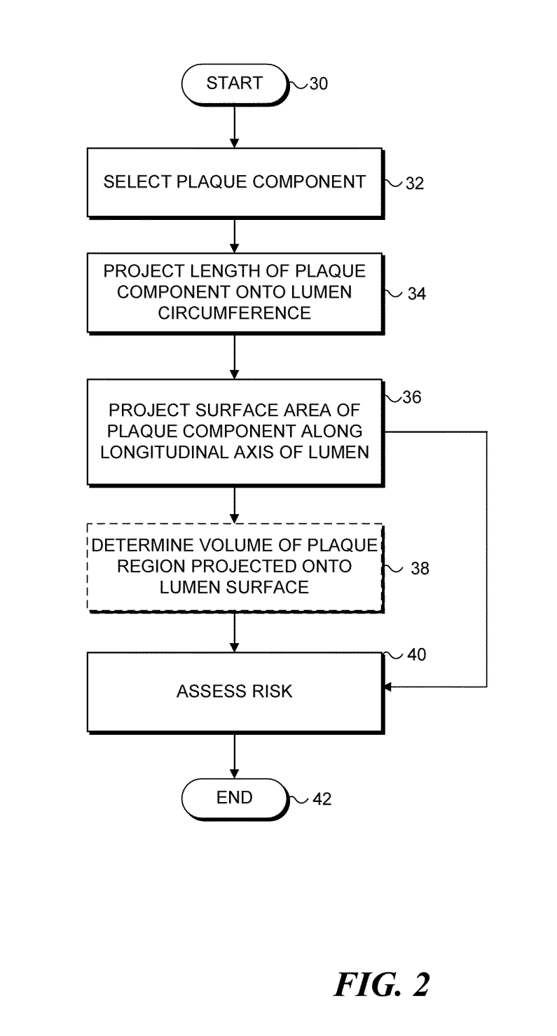

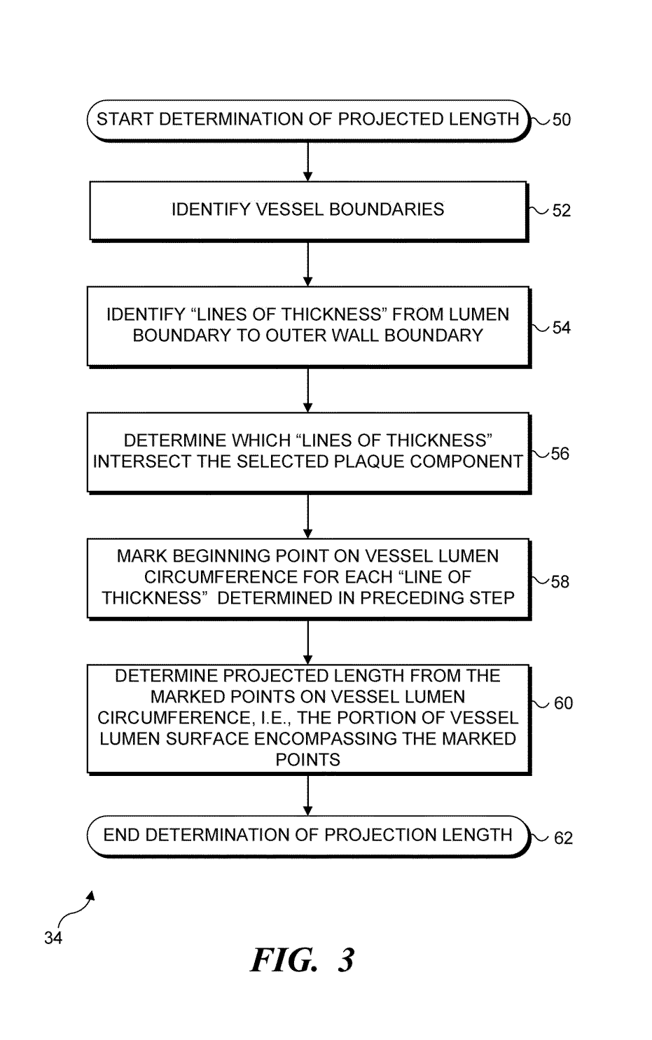

Exemplary overall steps of a method for carrying out the present approach are illustrated in FIG. 2. Details of the steps involved in determination of projection length, projection surface area, and projection volume are described in FIGS. 3-5. More specifically, returning to FIG. 2, the method begins at a step 30 and assumes that image data for a desired length of the artery being evaluated have been collected. In a step 32, the internal plaque component of interest that the risk assessment is to be based upon is selected. In this exemplary embodiment, the plaque component of interest is the LRNC. However, those skilled in the art will recognize that other plaque components could be projected, such as calcification 14 (see FIG. 1A) or a hemorrhage. In a step 34, a length of the selected internal plaque component projected onto the circumference of the vessel lumen is determined, for each cross-sectional image in the region of the vessel being evaluated. Next, in a step 36, a projected surface area (i.e., a shadow of the selected plaque component projected onto the longitudinal length of the vessel lumen) is determined by multiplying the plaque projection length by the thickness of each cross-sectional image or slice affected by the plaque in this region, and summing the results for all affected slices. At this point, risk can be assessed in a step 40. In the alternative, an optional step 38 can be carried out to provide a further value for assessing plaque vulnerability. In step 38, the projected volume is determined by multiplying the projected surface area by a thickness of the plaque component of interest, e.g., measured radially or in a direction outwardly from the surface of the vessel lumen. Risk can be assessed using this value also, in step 40. The method is then complete in a step 42.

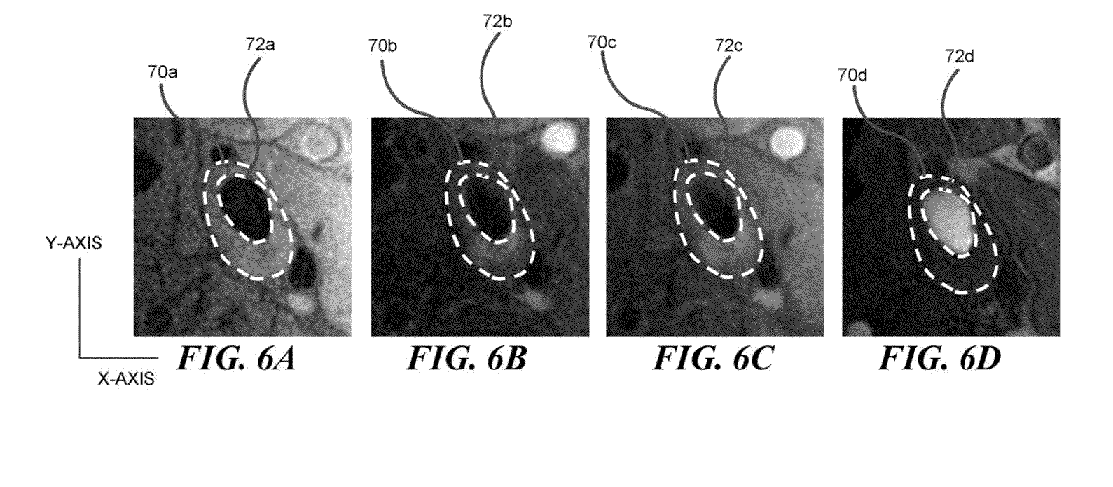

FIG. 3 provides details of step 34, for determining the length of a projection of the selected plaque component onto the circumference of the vessel lumen. The method begins in a start step 50. A step 52 provides for identifying the vessel boundaries. These vessel boundaries include the vessel lumen boundary, the outer wall boundary of the artery, and the boundaries of internal plaque components. In order to identify these vessel boundaries, serial, cross-sectional magnetic resonance (MR) images are acquired, as noted above. In other words, cross-sectional images are acquired generally perpendicular to the longitudinal axis (the Z axis) of the vessel. Multiple stacks of images are obtained for this vessel segment, where each stack is generated using different contrast weightings, i.e., by setting different MR acquisition parameters. Examples of these standard contrast weightings are shown in FIGS. 6A-6D that illustrate examples of T1-weighted, T2-weighted, proton-density-weighted, and time-of-flight images, respectively. Those skilled in the art will recognize that the cross-sectional images could also be obtained with ultrasound or CT imaging. After acquisition, the multiple contrast images for each cross-sectional slice or location are analyzed on a computer using software algorithms that identify the boundaries of the vessel wall and internal plaque components. For example, inner boundaries 70a-70d, and outer boundaries 72a-72d are identified in FIGS. 6A-6D. In this exemplary embodiment, plaque components are automatically identified using a morphology enhanced probabilistic plaque segmentation technique, such as described in commonly assigned co-pending U.S. Published patent application No. 20080009702, Ser. No. 11/445,510, filed Jun. 1, 2006, the specification and drawings of which are hereby specifically incorporated herein by reference.

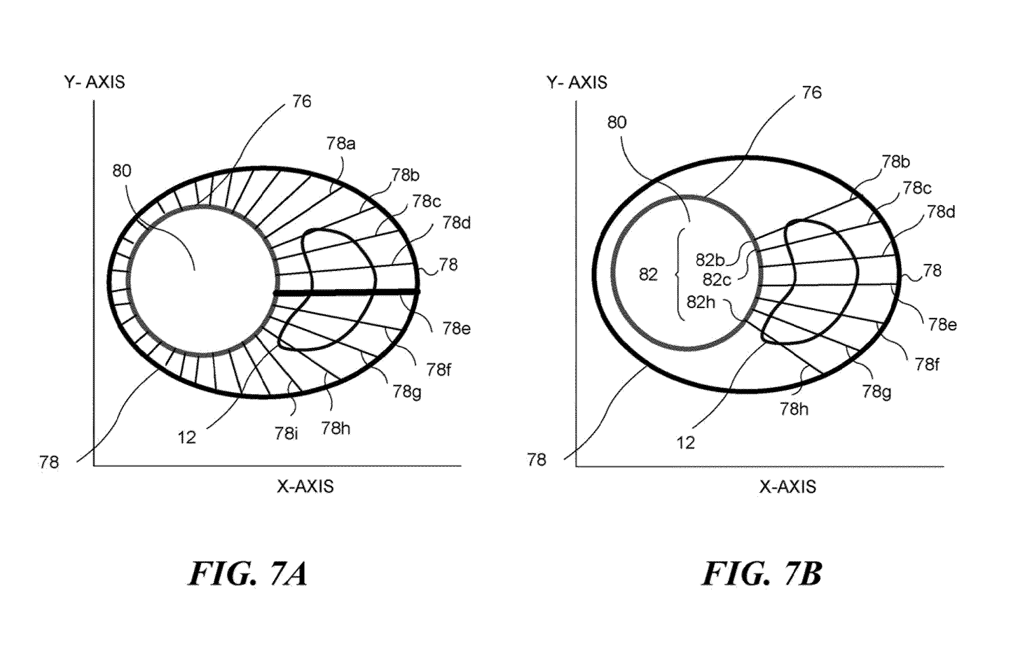

Once the boundaries are identified, a step 54 provides for identifying “lines of thickness” from the vessel lumen inner boundary to the artery outer wall boundary. A “line of thickness” is a line that connects a boundary to a different boundary. For example, FIG. 7A illustrates examples of lines of thickness 78a-78i that are drawn between a vessel lumen inner boundary 76 and an outer wall boundary 78 of a vessel 80 at a location i. A step 56 requires identification of the “lines of thickness” that intersect the selected plaque component. In other words, if a line connecting a boundary to a different boundary crosses over any portion of the selected plaque component, this “line of thickness” is considered to be effective in determining the projected length of the selected plaque component onto the lumen surface. In this exemplary embodiment, lines of thickness are determined using the method described in commonly assigned U.S. Pat. No. 7,353,117.

FIG. 7B illustrates lines of thickness that intersect a selected plaque component, such as LRNC 12. These are lines of thickness 78b-78h. For each effective line of thickness, a step 58 provides for marking its starting point on the boundary of the vessel lumen circumference. Therefore, for lines of thickness 78b-78h, their respective starting points 82b-82h, are marked on the vessel lumen surface boundary. Those skilled in the art will recognize that instead of using this approach to projecting a selected component onto the surface of a vessel lumen (i.e., the approach based on lines of thickness), any other standard projection technique can instead be used. For example (and without any implied limitation), the closest point on the vessel lumen boundary could be found for all points within a selected component in the plaque region.

A step 60 provides for determining the plaque projected length from all of the points of each effective line of thickness that are marked on the vessel lumen surface or perimeter, by adding the distances between each point on the circumference of the vessel lumen, which is equal to the portion of the vessel lumen perimeter that encompasses the points intersected by all of the lines of thickness identified in step 58. Thus, a set of marked points 82 is the projection of the selected component onto the vessel lumen, yielding a projection length, lₗ, for each cross-sectional slice, i. The method ends in a step 62.

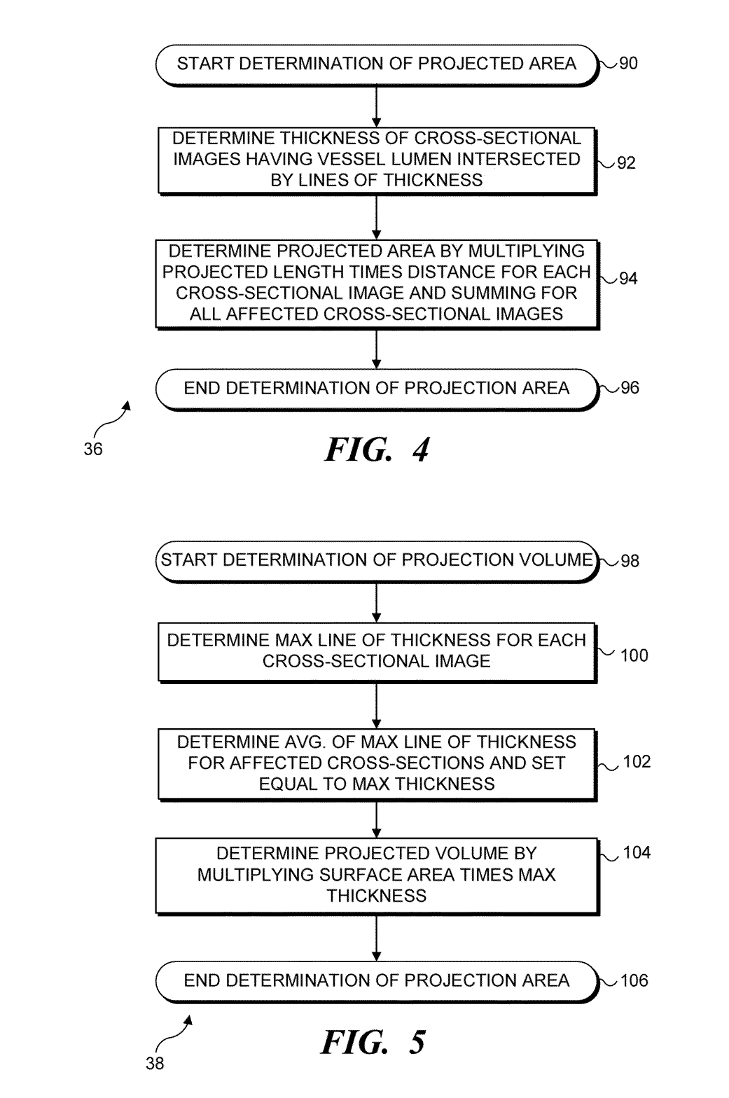

FIG. 4 illustrates details of step 36 (FIG. 2) relating to how the projected surface area of the selected plaque component onto the vessel lumen is determined. The method begins in a step 90. A step 92 is necessary to determine the spacing between adjacent, cross-sectional images. In a step 94, the projected surface area is calculated by multiplying the projected length on each cross-sectional image times this spacing, and then determining the sum of the resulting products (for all such cross-sectional images in which the vessel lumen is intersected by lines of thickness). In other words, the total projected surface area is computed by multiplying the total projected lengths of the selected plaque component for all cross-sections by the average spacing for all such cross-sectional images affected. The calculation is then completed in a step 96.

For example, the selected plaque component might be the LRNC. If the area measurement is limited to a 10 mm longitudinal segment of a vessel, the area of the LRNC projected on the vessel may be determined by:

$${A}_{projection} = \frac{10}{N} {max}_{j} {\sum }_{i = j}^{j + N - 1} {l}_{i} \qquad [ 1 ]$$

where N is the number of cross-sectional images spanning 10 mm. The “10” in the numerator of this example reflects the 10 mm of coverage, which was chosen to account for possible differences in coverage between different imaging protocols.

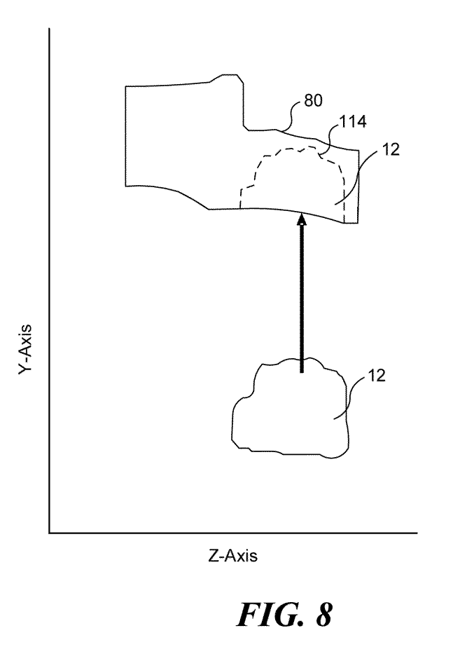

FIG. 8 illustrates the projection of an area 114 of a plaque component onto vessel lumen 80, along a portion of a longitudinal axis of a segment of the vessel. This surface area projection, in other words, this “shadow,” of the plaque component on vessel lumen 80 reflects its impact on the vessel lumen due to either the size of the plaque component or its proximity to the vessel lumen. Using this technique, changes in the projected area can be monitored during plaque progression to further assess the risk to the patient.

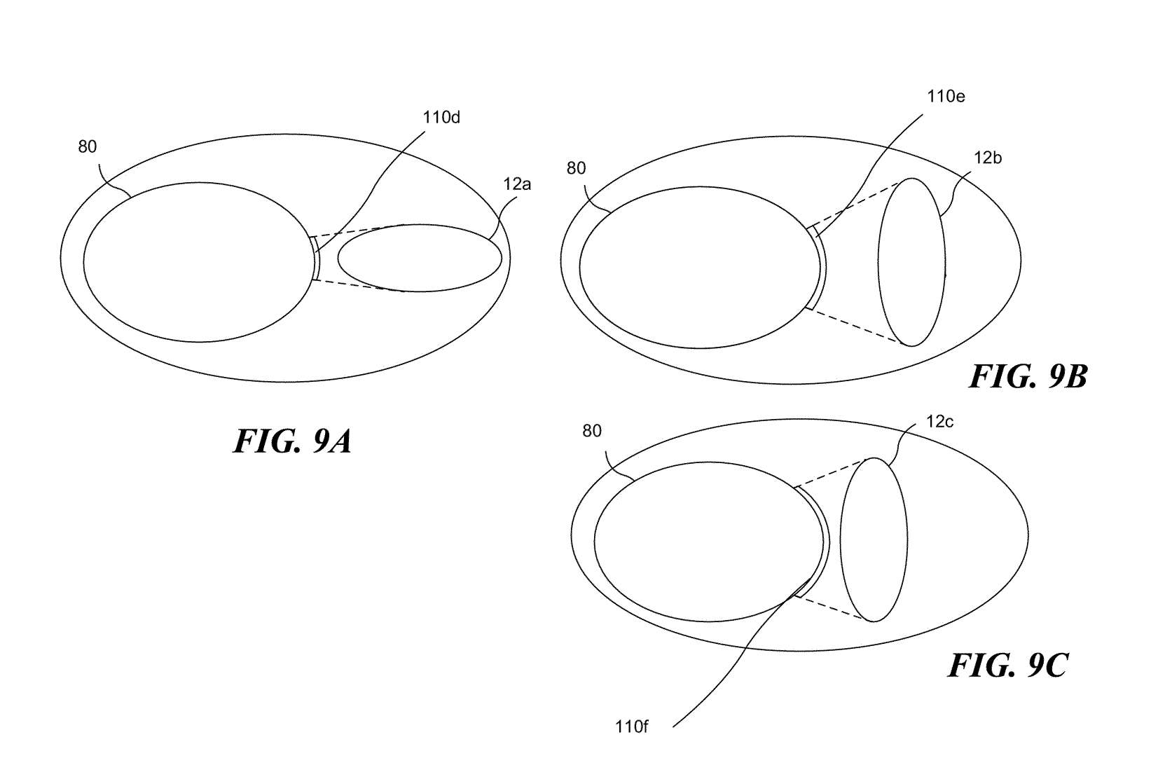

FIGS. 9A-9C illustrate the importance of measurements of the projected area of plaque components onto a vessel lumen for discerning the difference in plaque vulnerability when the plaque components have the same size in a cross-sectional view. These Figures illustrate vessel lumen 80 with three different projected lengths for LRNCs 12a-12c. Note that each of the LRNCs are of the same size, but are in a different orientation or distance away from vessel lumen 80. A visual comparison between the three Figures shows how projected lengths 110d-110f vary for each LRNC, as a result of the orientation and/or spacing between the vessel lumen and the LRNC. Although the size of LRNCs 12a-12c are the same, once the thickness of the affected cross-sectional images is multiplied by the sum of the respective projected lengths on each affected cross-sectional slice to determine the projected surface area, it is apparent that the projected area of the LRNC on the vessel lumen will also vary. Thus, these three plaque components will have a different impact on the vessel lumen in the analysis of the projected surface area, although the differences in the projected surface area may not initially appear evident simply from an inspection of the size of each LRNC in this example.

FIG. 5 illustrates details of step 38 (FIG. 2) for determining the total volume of a projection of the selected plaque component onto the vessel lumen. One previously developed technique that may be used for this step is disclosed in U.S. Pat. No. 7,353,117. Note that this is an optional step that can be used as a further indicator of risk or plaque vulnerability. The method begins in a step 98. A step 100 determines the maximum length of the lines of thickness, dᵢ^{max}, for each cross-sectional image within the longitudinal segment affected by the selected plaque component. The longitudinal segment of the vessel, which may be, for example, 10 mm in length. In a step 102, these maximum values are then averaged, and the average is set equal to the maximum thickness, d̅^{max}. For example, the maximum thickness for a 10 mm longitudinal segment of a vessel may be expressed as:

$${\overline{d}}^{max} = \frac{1}{N} {max}_{j} {\sum }_{i = j}^{j + N - 1} {d}_{i}^{max} \qquad [ 2 ]$$

where N is the number of slices spanning 10 mm. This value is the thickness used in the computing the projected volume in a step 104. Averaging over 10 mm is done in this example, to limit the impact of isolated measurement errors due to misplacement of vessel boundaries. Those skilled in the art will recognize that other standard thickness measurement technique (e.g. “centerline”) could be used to determine d̅^{max}. Furthermore, other alternatives to d̅^{max} include using the maximal wall thickness to compute the volume, or a measurement can be taken of the average distance of the projected surface area from the outer wall boundary of the vessel. Distance can also be measured to another point, such as from the vessel lumen boundary to the most distant boundary of the LRNC. Those skilled in the art will also realize that the computation could also be performed using individual cross-sections or segments of any length.

In step 104, the projected volume is determined by multiplying the projected surface area times the maximum thickness. Thus, multiply A_{projection} by d̅^{max} yields the “projected volume.” Reporting d̅^{max} in mm and A_{projection} in mm² yields a projected volume in units of mm³. The calculation is then complete in a step 106.

### Validation

To test whether the projected volume is associated with the risk of clinical events, the approach described above was applied to MRI data from a group of 46 subjects with known outcome (clinical event or no clinical event for 3 years). All subjects were recruited on the basis of having 50-79% carotid stenosis by duplex ultrasound and no prior cerebrovascular symptoms (stroke or transient ischemic attack). Eleven subjects developed symptoms later in the study.

MR images of the carotid arteries were collected with a standardized imaging protocol including T1, T2, proton density, and time-of-flight weightings as shown in FIGS. 6A-6D. These images were then analyzed by a trained radiologist using MRI-PlaqueView software of VPDiagnostics, Inc., Seattle, Wash. to delineate the boundaries of the vessel and plaque components on each cross-sectional image. These boundaries were used as the input to the system to compute the projected volume.

For subjects that remained asymptomatic, the vulnerable plaque volume averaged 98±130 mm³, whereas for symptomatic subjects the average value was 272±132 mm³, a difference that was highly significant based on a t-test (p<0.001). An optimal cutpoint of 150 mm³ produced a 91% sensitivity and 80% specificity for predicting which subjects would develop symptoms.

### System for Implementing the Present Invention

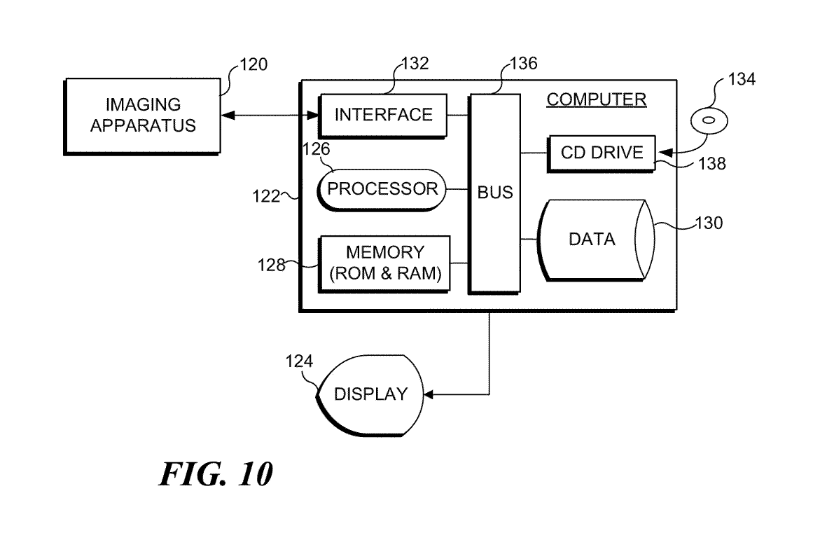

FIG. 10 schematically illustrates an exemplary system suitable for implementing the exemplary methods. The system includes a generally conventional imaging device 120, such as an MRI, ultrasound or CT imaging apparatus that is connected to a computer 122 (or other type of computing device). Computer 122 may, for example, be a generally conventional personal computer, or a dedicated controller specifically intended for controlling imaging device 120. It will be understood that some other form of programmable or hardwired logic device that is configured to control implementation of the steps comprising the present novel approach described herein might be used instead of computer 122. Details of the imaging device need not be and are not specifically illustrated or discussed herein.

Computer 122 is coupled to a display 124, which is used for displaying MRI images or ultrasound or CT images to an operator. Included within computer 122 is a processor 126. A memory 128 (comprising both read only memory (ROM) and random access memory (RAM)), a non-volatile storage 130 (such as a hard drive or other non-volatile data storage device) for storage of data, digital signals, and software programs, an interface 132, and an optional optical drive 134 are coupled to processor 126 through a bus 136. Optical drive 134 is not essential, but may be desirable for reading a compact disk (CD) 138 (or other optical storage media) on which machine instructions are stored for implementing the present invention and other software modules and programs that may be run by computer 122. The machine instructions are loaded into memory 128 before being executed by processor 126 to carry out the steps of the present novel approach.

### Validation

The capability of this system for estimating vulnerable plaque volume that may be indicative of risk to a patient was based on choosing parameters that can be accurately and reproducibly measured. With improvements in technology, this system lends itself to a wide variety of alternatives on the same basic theme, many of which have been set forth as alternatives described above. It is also contemplated that additional parameters, such as additional measurements of other parameters that are also associated with risk could be added. For example, the thickness of the fibrous layer separating the plaque from the vessel lumen, which should be proportional to the strength of the layer could be taken into account.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for assessing a risk of atherosclerosis in an artery, based on a plurality of images of the artery, comprising the steps of:
(a) selecting a plaque component in the plurality of images;
(b) projecting a dimension of the plaque component onto a circumference of a lumen within the artery;
(c) projecting a surface area of the plaque component along a longitudinal axis of the lumen; and
(d) assessing the risk of atherosclerosis in the artery as a function of the dimension of the plaque component projected onto the circumference of the lumen, and the surface area of the plaque component projected along the longitudinal axis of the lumen.

2. The method of claim 1, further comprising the steps of:
(a) determining a region of the plaque component;
(b) projecting the region of the plaque component onto a surface of the lumen to define a corresponding volume; and
(c) carrying out the step of assessing the risk of atherosclerosis also based on the corresponding volume of the region of the plaque component projected onto the surface of the lumen.

3. The method of claim 1, wherein the step of projecting the dimension of the plaque component onto the circumference of the lumen comprises the steps of:
(a) identifying boundaries of the artery;
(b) identifying lines of thickness between an internal boundary of a wall of the artery and an outer boundary of the wall of the artery;
(c) determining specific lines of thickness that intersect the plaque component;
(d) marking points where each specific line of thickness intersects the circumference of the lumen; and
(e) determining a length along the circumference resulting from the step of projecting the dimension of the plaque component to encompass the points marked on the circumference of the lumen.

4. The method of claim 3, wherein the step of projecting the surface area of the plaque component along a longitudinal axis of the lumen comprises the steps of:
(a) determining a thickness of each cross-sectional image in which the lumen is intersected by the lines of thickness;
(b) determining a projected area of the plaque component for each cross-sectional image intersected by the lines of thickness;
(c) for each cross-sectional image intersected by the lines of thickness, multiplying a projected length of the plaque component by the thickness of the cross-sectional image to determine a projected area for each such cross-sectional image; and
(d) determining a sum of all projected areas for the cross-sectional images.

5. The method of claim 4, wherein the step of projecting the region of the plaque component onto the surface of the lumen to define the corresponding volume comprises the steps of:
(a) determining a maximum line of thickness for each cross-sectional image in which the plaque component is disposed;
(b) determining an average maximum line of thickness based on the maximum line of thickness determined and set equal to a maximum thickness; and
(c) determining a projected volume of the region of the plaque component by multiplying the surface area projected along the longitudinal axis of the lumen by the maximum thickness.

6. The method of claim 1, further comprising the step of producing the images by at least one step selected from the group of steps consisting of:
(a) imaging the artery to form successive cross-sectional images along a longitudinal extent of the artery using magnetic resonance (MR) imaging;
(b) imaging the artery to form successive cross-sectional images along a longitudinal extent of the artery using computed tomography imaging; and
(c) imaging the artery to form successive cross-sectional images along a longitudinal extent of the artery using ultrasound imaging.

7. The method of claim 6, wherein the step of imaging using MR imaging comprises the step of employing different MR acquisition parameters for creating a plurality of sets of MR images of the artery, to achieve different contrast weightings for each set of MR images, for use in assessing the risk of atherosclerosis in the artery.

8. The method of claim 1, wherein the step of selecting the plaque component comprises the step of selecting either a lipid-rich necrotic core, a calcification, or a hemorrhage in a wall of the artery.

9. A non-transitory medium storing machine readable instructions that are executable by a computing device to facilitate assessing a risk of atherosclerosis in a artery, based on a plurality of images of the artery, the machine readable instructions being operable to carry out a plurality of functions, including:
(a) selecting a plaque component in the plurality of images;
(b) projecting a dimension of the plaque component onto a circumference of a lumen within the artery;
(c) projecting a surface area of the plaque component along a longitudinal axis of the lumen; and
(d) assessing the risk of atherosclerosis in the artery as a function of the dimension of the plaque component projected onto the circumference of the lumen, and the surface area of the plaque component projected along the longitudinal axis of the lumen.

10. The non-transitory medium of claim 9, wherein the plurality of functions further include:
(a) determining a region of the plaque component;
(b) projecting the region of the plaque component onto a surface of the lumen to define a corresponding volume; and
(c) assessing the risk of atherosclerosis also based on the corresponding volume of the region of the plaque component projected onto the surface of the lumen.

11. The non-transitory medium of claim 9, wherein the function of projecting the dimension of the plaque component onto the circumference of the lumen is implemented by:
(a) identifying boundaries of the artery;
(b) identifying lines of thickness between an internal boundary of a wall of the artery and an outer boundary of the wall of the artery;
(c) determining specific lines of thickness that intersect the plaque component;
(d) marking points where each specific line of thickness intersects the circumference of the lumen; and
(e) determining a length along the circumference resulting from the step of projecting the dimension of the plaque component to encompass the points marked on the circumference of the lumen.

12. The non-transitory medium of claim 9, wherein the function of projecting the surface area of the plaque component along a longitudinal axis of the lumen is implemented by:
(a) determining a thickness of each cross-sectional image in which the lumen is intersected by the lines of thickness;
(b) determining a projected area of the plaque component for each cross-sectional image intersected by the lines of thickness;
(c) for each cross-sectional image intersected by the lines of thickness, multiplying a projected length of the plaque component by the thickness of the cross-sectional image to determine a projected area for each such cross-sectional image; and
(d) determining a sum of all projected areas for the cross-sectional images.

13. A system for use in automatically assessing a risk of atherosclerosis in an artery, based on a plurality of images of the artery, comprising:
(a) a memory in which are stored machine instructions;
(b) a user input device;
(c) a display on which text and graphics are displayed; and
(d) a hardware processor that is coupled to the memory, the user input device, and the display, the processor executing the machine instructions stored in the memory to carry out a plurality of functions, including:
(i) selecting a plaque component in the plurality of images;
(ii) projecting a dimension of the plaque component onto a circumference of a lumen within the artery;
(iii) projecting a surface area of the plaque component along a longitudinal axis of the lumen; and
(iv) assessing the risk of atherosclerosis in the artery as a function of the dimension of the plaque component projected onto the circumference of the lumen, and the surface area of the plaque component projected along the longitudinal axis of the lumen.

14. The system of claim 13, wherein execution of the machine instructions by the processor further causes the following functions to be executed:
(a) determining a region of the plaque component;
(b) projecting the region of the plaque component onto a surface of the lumen to define a corresponding volume; and
(c) assessing the risk of atherosclerosis also based on the corresponding volume of the region of the plaque component projected onto the surface of the lumen.

15. The system of claim 13, wherein execution of the machine instructions causes the processor to project the dimension of the plaque component onto the circumference of the lumen by:
(a) identifying boundaries of the artery;
(b) identifying lines of thickness between an internal boundary of a wall of the artery and an outer boundary of the wall of the artery;
(c) determining specific lines of thickness that intersect the plaque component;
(d) marking points where each specific line of thickness intersects the circumference of the lumen; and
(e) determining a length along the circumference resulting from the step of projecting the dimension of the plaque component to encompass the points marked on the circumference of the lumen.

16. The system of claim 15, wherein execution of the machine instructions further causes the processor to project the surface area of the plaque component along a longitudinal axis of the lumen by:
(a) determining a thickness of each cross-sectional image in which the lumen is intersected by the lines of thickness;
(b) determining a projected area of the plaque component for each cross-sectional image intersected by the lines of thickness;
(c) for each cross-sectional image intersected by the lines of thickness, multiplying a projected length of the plaque component by the thickness of the cross-sectional image to determine a projected area for each such cross-sectional image; and
(d) determining a sum of all projected areas for the cross-sectional images.

17. The system of claim 16, wherein execution of the machine instructions causes the processor to project the plaque component onto the surface of the lumen to define the corresponding volume, by implementing the following functions:
(a) determining a maximum line of thickness for each cross-sectional image in which the plaque component is disposed;
(b) determining an average maximum line of thickness based on the maximum line of thickness determined and set equal to a maximum thickness; and
(c) determining a projected volume of the region of the plaque component by multiplying the surface area projected along the longitudinal axis of the lumen by the maximum thickness.

18. The system of claim 13, wherein execution of the machine instructions further causes the processor to produce the images by implementing at least one function selected from the group of functions consisting of:
(a) imaging the artery to form successive cross-sectional images along a longitudinal extent of the artery using an magnetic resonance (MR) imaging system;
(b) imaging the artery to form successive cross-sectional images along a longitudinal extent of the artery using a computed tomography imaging system; and
(c) imaging the artery to form successive cross-sectional images along a longitudinal extent of the artery using an ultrasound imaging system.

19. The system of claim 18, wherein when using the MR imaging system, the machine instructions cause the processor to employ different MR acquisition parameters for creating a plurality of sets of MR images of the artery, to achieve different contrast weightings for each set of MR images, for use in assessing the risk of atherosclerosis in the artery.

20. The system of claim 13, wherein the execution of the machine instructions causes the processor to select the plaque component by selecting either a lipid-rich necrotic core, a calcification, or a hemorrhage in a wall of the artery.
